(19) United States
(12) Patent Application Publication
(10) Pub. No.: US 2023/0226133 A1
(43) Pub. Date: Jul. 20, 2023

(54) METHOD FOR EXTRACTING CERAMIDE-CONTAINING MATERIAL OR CERAMIDE FROM WHOLE APPLES AND/OR APPLE JUICE EXTRACTION RESIDUE, AND COMPOSITION INCLUDING SAID CERAMIDE

(71) Applicants: NIHON HARUMA KABUSHIKI KAISHA, Aomori (JP); HIROSAKI UNIVERSITY, Aomori (JP)

(72) Inventors: Tsuyoshi Narumi, Aomori (JP); Junji Ichita, Aomori (JP); Kou Itoku, Aomori (JP); Naoki Nanashima, Aomori (JP); Akira Nakajima, Aomori (JP)

(21) Appl. No.: 18/010,833

(22) PCT Filed: Jun. 17, 2021
(86) PCT No.: PCT/JP2021/023075

§ 371 Date: Dec. 16, 2022

(30) Foreign Application Priority Data
Jun. 19, 2020 (JP) 2020-106402

(51) Int. Cl. A61K 36/73; A61K 31/164; A61P 25/28

(52) U.S. Cl. CPC A61K 36/73; A61K 31/164; A61P 25/28; A61K 2236/333; A61K 2236/51; A61K 2236/53

(57) ABSTRACT

A method for extracting a ceramide-containing material or ceramide from whole apples and/or apple juice extraction residue. Furthermore, to ensure utility as a food, especially a functional food, or a raw material for a cosmetic or medicine, that includes the ceramide-containing material. [Solution] In the present invention, an adsorbent is added to an ethanol treatment solution of whole apples and/or apple juice extraction residue, the mixture is stirred and subjected to solid-liquid separation, and solid material precipitated from the solution is separated, after which water is added to the resultant concentrate and the temperature thereof is maintained, whereby the resultant settled component is recovered and dried to obtain a ceramide-containing material or ceramide. Using the ceramide obtained from whole apples and/or apple juice extraction residue, there are formed a composition for promoting production of hyaluronic acid and/or type II collagen, and a composition for promoting improvement of memory impairment and/or enhancement of the level of phosphorylation of extracellular signal-related kinases (ERK1/2).

### TECHNICAL FIELD

The present invention relates to a method/process for extracting apple-derived ceramide-containing matter or apple-derived ceramide from whole apples and/or apple juice extraction residues and, more particularly, an extraction process combined with fractionation of ursolic acid (also called ulsol acid) and a polyphenol solution.

The present invention also relates to use of ceramide as foodstuffs inclusive of functional foodstuff in particular, cosmetics and pharmaceuticals. More specifically, the present invention is concerned with a composition containing ceramide obtained from whole apples and/or apple juice extraction residues.

It is here understood that the “ceramide” and “ceramide-containing matter” referred to herein are derivable from apples.

### BACKGROUND OF THE INVENTION

Apple fruit, irrespective of its kind, includes not only dietary fibers inclusive of pectin and various useful ingredients such as ceramide, ursolic acid or polyphenols but also contains potassium in abundance. When they are ingested as foodstuffs, there are some reports about various effects of these ingredients working on nutrition intake effects or, possibly, health maintenance effect or disease prevention effect.

In recent aging society in particular, there is mounting demand for improving the quality of life while maintaining beauty and health, which demand could be satisfied by extracting ingredients contained in apple fruits and using them as a raw material for secure foodstuffs inclusive of functional foodstuffs, cosmetics or medicals.

Apple juice is generally produced by way of steps of using post-harvested whole apples as raw material, washing the whole applies, and pulverizing and pressing them for removal of juice ingredients. Incidentally, apple juice extraction residues, irrespective of useful ingredients contained in the initial whole apple remaining, are characterized by rapid rotting and difficult storage, leading to generation of industrial wastes as well as air pollution and environmental pollution with the result that wastes must be disposed at increased costs and in much labor. If useful ingredients are extracted out of apple juice extraction residues to determine their functionality scientifically, they are expected to be used as a raw material for foodstuffs in general and functional foodstuffs in particular as well as cosmetics or medicals with reduced costs for waste disposals.

### PRIOR ARTS

### Patent Documents

Patent Document 1: Japanese Patent Application Publication No. 2015-57378A

Patent Document 2: Japanese Patent Application Publication No. 2019-154296A

Patent Document 3: Japanese Patent Application No. 2020-24773

Patent Document 4: Japanese Patent Application No. 2020-510057

With respect to the present invention, there are some prior arts.

Patent Document 1 is a prior patent application specification published under the inventor names of the present invention yet unexamined. Patent Document 1 discloses a process of using whole apples and/or apple juice extraction residues as a raw material, comprising a common step 1 for water treatment and drying treatment followed by a common step 2 in which ethanol extraction is carried out and which comprises a pectin step of extracting pectin out of dried matter obtained after ethanol treatment and a ceramide step of extracting ceramide out of an ethanol treated solution. On the contrary, the present invention provides a process of extracting ceramide-containing matter or ceramide of an ethanol-treated solution, a process of recovery of ethanol, offers reference to the effect of the ceramide-containing matter on cultured cells derived from the skin or cartilage, and discloses a process of extracting ursolic acid or a polyphenol solution in the step of extracting ceramide-containing matter.

Patent Document 2 discloses a process of efficient concentration of glycosylceramide derived from apples by applying heat and enzyme to apples or their extraction residues, but it does neither say or suggest about anything about how to extract ursolic acid or a polyphenol solution other than the ceramide derived from apples.

Patent Document 3 discloses a process of production of foodstuffs while making use of extraction of ingredients derived out of a citrus fruit or its pericarp by applying Japanese spirits (or shochu) or vinegar, but it does neither say or suggest about how to extract ceramide, ursolic acid or a polyphenol solution derived from apples, and anything about the effect of ceramide-containing matter on dementia models.

Patent Document 4 shows that nicotine, nobiletin, sinesechin and/or chinpi providing a medical composition effective for the treatment of central neurodegenative diseases is confirmed by experimentation using animals and/or cultured cells, but it does neither say nor suggest about how to extract ceramide, ursolic acid or a poly-phenol solution derived from apples, and anything about the effect of ceramide-containing matter on dementia models.

### NON-PATENT DOCUMENTS

Non-Patent Document 1: “Functional evaluation of ceramide derived from peaches” (Yuki Kobayasshi et al, Food Style 21, vol. 19, pp. 22-26 (2015)

Non-Patent Document 2: “Preventive Action of Nobiletin, A Constituent of Aurant II Nobilis Pericarpim with Anti-dementia Activity, against Amyloid-β Peptid—induced Neurotoxicity Expression and Memory Impairment” (Tohru Yamaguni et al., Yakugaku Zasshi, vol. 130, pp.517-520 (2010)

Non-Patent Document 3: T. Maurice, M. Hiramatshu, J. Itoh, T. Kameyama, T. Hasegawa, and T. Nabeshima, Brain Res., vol. 647, pp. 44-56 (1994)

Non-Patent Document 4: Y-J, Huang, C-H. Lina, H-Y. Lanea and G. E. Tsai, Current Neuropharmacology, vol. 10, pp. 272-285 (2012)

Non-Patent Document 5: J. P. Adams and J. D. Sweatt, Annu. Rev. Pharmacol. Toxicol., vol 42, pp. 135-163 (2002)

Non-Patent Document 1 provides such a disclosure as mentioned below: it first refers to a hypothesis that transdermal application of peach-derived ceramide brings about an improvement in the moisturizing effect and barrier function of the skin because addition of peach-derived ceramide to cultured cells derived out of the human skin induces an increase in the amount of ceramide contained in the cells. Further, it goes on that as a result of oral absorption of peach-derived ceramide in a human, there was an increase found in the amount of moisture transpiration of the skin, leading to an increase in the amount of moisture in a stratum corneum and an improvement in skin texture; as was the case with transdermal administration, oral administration was also expected to induce an improvement in the moisturizing effect and barrier function of the skin. The peach-derived ceramide is composed of plural ceramide ingredients as is the case with ceramides derived out of other plants, with its sphingoid base being similar to that of rice, corn, and conjak, and/or its fatty acid ingredient being similar to that of an apple. Ceramide derived out of plants such as rice, corn, and conjak was found to induce an improvement in the moisturizing effect of the skin via oral ingestion or transdermal administration, and the peach-derived ceramide was confirmed to have a similar moisturizing effect as well.

Non-Patent Document 2 discloses that with clarification of the pathological onset function of cognitive impairments that increase recently in case numbers, a method of estimation using an experimental animal or cultured cells providing such disease models was well established, going on that nobiletin contained in citrus fruits was found to have a function of preventing and mitigating the onset and symptom of them. The ceramide-containing matter derived out of an apple disclosed herein is an ingredient different from nobiletin derived from citrus fruits with the mechanism of action different from that of the dementia model used herein.

### SUMMARY OF THE INVENTION

Objects of the Invention

The invention disclosed herein has one object to provide a method/process of extracting ceramide-containing matter or ceramide out of whole apples and/or apple juice extraction residues. In addition, the present invention discloses that said ceramide-containing matter has an effect on cultured cells derived out of the human skin or cartilage with demonstration of that effect having on dementia model mouse and/or the neural model cells derived out of rats, and has another object to use it as a material for foodstuffs in general, and healthy foodstuff, cosmetics or pharmaceuticals.

### How to Accomplish the Objects

The invention disclosed herein provides a method/process of extracting ceramide-containing matter or ceramide out of whole apples and/or apple extraction residue. In this connection, the present invention discloses an effect of the ceramide-containing matter on cultured cells derived out of the skin or cartilage, and an effect of the ceramide-containing matter on dementia model mice and/or neural model cells derived from rats. In addition, ursolic acid or a polyphenol solution is each obtained in the aforesaid extraction method/process. The present invention also discloses a method/process of recovering ethanol for reuse, as detailed below.

FIG. 1 shows a step of extracting ceramide-containing matter out of whole apples and/or apple extraction resides in the form of Product 1. The common step referred in FIG. 1 means a pretreatment step common to the ceramide step and pectin step disclosed in the invention of the prior patent application. In FIG. 1, the expression written in a rectangle depicted by a fine line stands for a step whereas the expression written in a rectangle depicted by a thick line stands for a substance. More specifically, the method/process of the invention disclosed herein comprises a step (common step 1 and/or common step 2 in FIG. 1) in which an ethanol treatment solution and post-ethanol treatment dried matter are obtained by way of common step 1 in which after treatment of whole apples and/or apple juice extraction residues with water, drying treatment is applied, and common step 2 in which ethanol is added to water-treated, dried matter for extraction and solid-liquid separation plus

Step (A) in which an adsorbent is added to the aforesaid ethanol solution for solid-liquid separation thereby removing the adsorbent for obtain an adsorbent-treated solution (ceramide step 1 in FIG. 1 and/or FIG. 9,
Step (B) in which the adsorbent-treated solution is let standing for solid-liquid separation of precipitating matter thereby obtaining a solution from which precipitates are removed (precipitate-free liquid, ceramide step 2 in FIG. 1 and/or FIG. 10),
Step (C) in which the aforesaid precipitate-free liquid is concentrated under reduced pressures to obtain a concentrate and recover ethanol (ceramide step 3 in FIG. 1 and/or FIG. 11), and
Step (D) in which water is added to the aforesaid concentrate while carrying out heat insulation for solid-liquid separation of precipitating matter followed by drying thereby obtaining ceramide-containing matter as a solid ingredient (ceramide step 4 in FIG. 1 and/or FIG. 12).

Referring to the effect of the ceramide-containing matter extracted by the foregoing method/process on cultured cells derived out of the skin or cartilage, the present invention discloses:

Effect (E) of the ceramide-containing matter on the production of hyaluronic acid in the cultured cells derived from the human skin (FIG. 2 and/or FIG. 3), and
Effect (F) of the ceramide-containing matter on the production of type II collagen in the cultured cells derived from the human cartilage (FIG. 4 and/or FIG. 5).

Referring further to the effect of the ceramide-containing matter extracted by the foregoing method/process on dementia model mice and/or neural model cells derived from rats, the present invention discloses:

Effect (G) of the ceramide-containing matter on dementia model mice with memory impairments (FIG. 6), and
Effect (H) of the ceramide-containing matter on modulation of neuronal signal transduction in neural model cells derived from rats (FIG. 7 and/or FIG. 8).

Referring further to each of the ceramide step 1 to ceramide step 4, the present disclosure teaches that

(I) In ceramide step 2, the solid matter precipitated from the adsorbent treatment liquid upon let standing is ursolic acid (ceramide step 2 and FIG. 9),
(J) In ceramide step 3, the precipitate-free liquid can be concentrated under reduced pressures for recovery and reuse of ethanol (ceramide step 3 and FIG. 10), and
(K) In ceramide step 4, the solution obtained by adding water to the concentrate followed by heat insulation for precipitation and solid-liquid separation of the ceramide-containing matter is a polyphenol solution (ceramide step 4 and FIG. 11)

Still further, the present disclosure shows (L) the separation and purification of ceramide from the ceramide-containing matter extracted in the aforesaid ceramide steps 1 to 4 (FIG. 12).

FIG. 14 shows the whole method/process comprising the step of extracting ceramide-containing matter from the whole apples and/or apple juice extraction residues in the form of Product 1 and the intermediate steps including ceramide step 2 of obtaining ursolic acid from ceramide step 2 in the form of Product 2, recovered ethanol from ceramide step 3 and a polyphenol solution from ceramide step 4 in the form of Product 3. The steps from ceramide step 1 to ceramide step 4 are a multi-stage method/process characterized in that each product is obtained by fractionation in each step. In FIG. 14, post-separation products are shown with the liquid ingredient on the left side and the solid matter on the right side.

The term “whole apple” used herein is understood to stand for all as-harvested apples inclusive of not only apple products put on the market for being eaten raw or processing irrespective of full ripeness or unripeness, but also apple products that retain original form like as-yet-not-processed (extracted) apples. That is, the “whole apples” refer to all as-harvested apples that retain original form like fallen apples, unripe apples, and apples excluded out of a sorting process. In other words, the “whole apples” refer to all apples that are not subjected whatsoever to any special processing resulting in deformation after harvesting.

The term “apple juice extraction residues” is understood to mean a solid portion of an apple fruit separated from a liquid portion by a known process such as crushing, grinding or pressing or, simply, an apple in a crushed or ground state. In one specific example, an apple fruit is crushed by means of a hammer crusher or the like into a size of about 5 to 30 mm, and then pressed down at a pressure of about 5 kg/cm² to 20 kg/cm²; however, it is noted that depending on type, apple fruits vary largely in state, so there is no particular limitation on the degree of crushing or grinding. In some cases, reliance is upon not only crushing treatment (where apple fruits are pulverized as by a hammer crusher to a size of about 5 to 30 mm) or grinding treatment (where apple fruits are pulverized to a size of about 5 mm or less as by a colloid mill or pulper but also disintegrating treatment (where coagulated solid matter is disintegrated as by a mesh or screen). In addition, the “apple juice extraction residues” used herein are understood to include not only each or all of “extraction residues” obtained by extracting treatment in an extraction plant but also the so-called “apple processing byproducts” occurring in extraction, juice and processing plants or factories inclusive of skins or cores discharged in cut apple production processes.

On the other hand, the term “ceramide(s)” is understood to mean glucosylceramide having glucose bonded to ceramide or galactosylceramide having galactose bonded to ceramide rather than a ceramide compound in which sphingosin and fatty acids are combined via amide bonding; these ceramides are also called cerebrosides. What is called ceramides derived from plants such as apples is present in this cerebroside state; however, as the name “ceramide” is generally available, the present disclosure follows precedent too. That is, the ceramide used herein is understood to stand for just only a ceramide compound wherein sphingosin and fatty acids are combined via amide bonding but also a cerebroside having glucose or galactose bonded to this ceramide compound.

It is also noted that the term “hyaluronic acid enhanced or increased in terms of production” or one type of poly-saccharide called mucopolysaccharide is typically widely distributed in connective tissues such as an eyeball vitreous body, an umbilical cord, a joint bone sap, blood vessel closing or the gastric mucosa. Hyaluronic acid is water soluble and high in terms of water retention with its aqueous solution being viscous.

In addition, collagen that is enhanced or increased in terms of production as described herein or one type of proteins present in animals is a fibrous ingredient or component present in bones, cartilage, tendons or the skin. Type II collagen is primarily found in cartilage.

N-methyl-D-aspartate receptor capable of recovering or getting back if impeded as described herein is understood to stand for a receptor that is found in animal neurons, taking part in memory, learning or neuronal cell death. Reportedly, an impediment to this receptor gives rise to memory impairment observed in Alzheimer's type dementia or other type dementias (see Non-Patent Documents 3 and 4).

Further, ERK enhanced as described herein is Extra-cellular Signal-regulated Kinase 1/2, standing for a signaling factor widely observed in life. As a cell receives a signal from outside, ERK1/2 is phosphorylated into an active body. Referring here to nerve cells, ERK1/2 is known to take part in memory formation with activation of ERK1/2 leading to symptom alleviation or treatment of dementias (see Non-Patent Document 5).

To add to this, MEK (MAPK/ERK Kinase) increased (as described herein) an enzyme working on phosphorylation of said ERK1/2. MEK/ERK signaling system is a signaling path that is known to act upstream of ERK1/2 to play an important role in introduction of extracellular signals into cell response. Referring here to a neural cell, MEK and ERK1/2 is known to take part in memory formation, leading to symptom alleviation and/or treatment of dementias (see Non-Patent Document 5).

Ursolic acid (also called ulsol acid) used herein is a five-membered ring triterpene-based carboxylic acid contained in plants, and refers to a compound contained in wax-like substances of a variety of trees in general and fruits and/or leaves in particular.

In addition, the term “polyphenol” used herein refers to pigment compounds and/or bitter ingredients widely distributed in plants. There are a plurality of polyphenol compounds mixed together in apple fruits. The pigment compound is a compound that constitutes the pigments of apple fruit skin and/or an apple fruit and consists of anthocyanins and/or glycosides thereof, with types of constituting compounds varying dependent on apple type. The polyphenol component of red pigments found in apple fruit skins consists mainly of cyanidin-3-glucoside.

There is a polyphenol called procyanidin also contained in an apple, which is a compound providing a bitter ingredient, and exists in the form of a polymer mixture of catechins.

Shown in the present disclosure is a whole process that comprises steps from ceramide steps 1 through 4 of extracting ceramide-containing matter that is a useful substance contained in an apple fruit from the whole apples and/or apple juice extraction residues. Referring to ceramide step 2 or ceramide step 4, the step of extracting ursolic acid and/or a polyphenol solution is explained. The ceramide and/or ursolic acid and/or poly-phenol are a general term for known compounds and/or compounds widely distributed in plants inclusive of apples, and an apple fruit is characterized by containing all useful such ingredients. In addition, the present invention makes it possible to extract these useful substances from raw materials or residues that are discharged industrially in large amounts such as whole apples and/or apple juice extraction residues.

### Advantages of the Invention

The present invention provides means or methods/processes for extracting ceramide-containing matter from whole apples and/or apple juice extraction residues. The present invention also shows that the foregoing ceramide-containing matter has an effect on cultured cells derived out of the human skin or cartilage. In the step of extracting the ceramide-containing matter, further, ursolic acid and a polyphenol solution are obtained with such effects or advantages as generally stated below.

### Effect 1

By virtue of the present invention, it is possible to extract ceramide-containing matter as Product 1 from whole apples and/or apple juice extraction residues, and by carrying out the present invention, it is possible to make effective use of unused material with its putrid smells contaminating or polluting environments in or around apple juice factories and cut down the cost of disposing residues that are treated as waste.

### Effect 2

As the ceramide-containing matter is added to cultured cells derived out of the human skin, it causes the amount of hyaluronic acid produced by the cells to be increased with an enhancement in the gene expression of hyaluronic acid synthesis enzyme.

### Effect 3

As the ceramide-containing matter is added to cultured cells derived from the human cartilage, it causes the amount of type II collage produced by the cells to be increased with an enhancement in the gene expression of producing type II collagen.

### Effect 4

According to a composition containing ceramide or ceramide-containing matter of the invention, memory impairments are improved, and/or the phosphorylation of ERK that is a signaling factor is increased.

### Effect 5

In the present invention, an adsorbent is added to an ethanol treatment liquid obtained by ethanol extraction of whole apples and/or apple juice extraction residues, resulting in decoloration of the solution and removal of odors, and precipitation and separation of ursolic acid as Product 2.

### Effect 6

In the present invention, the ethanol treatment liquid of whole apples and/or apple juice extraction residues is treated with an adsorbent, after which a solution from the precipitating ursolic acid is condensated under reduced pressures to obtain a concentrate of ceramide-containing matter and recovery of ethanol for reuse.

### Effect 7

In the present invention, water is added to the foregoing concentrate followed by heat insulation (heat-retaining) whereby a polyphenol solution can be extracted as Product 3 in a solution from which solid ceramide-containing matter is removed.

### Effect 8

In the present invention, ceramide can be separated and purified from the foregoing ceramide-containing matter.

### Effects Attainable from the Respective Inventions of the Claims

According to claim 1, the ceramide-containing matter can be extracted as Product 1 of the liquid of whole apples and/or apple juice extraction residues treated with ethanol.

According to claim 2 wherein the production of hyaluroic acid and type II collagen by human cells can be enhanced by addition of ceramide-containing matter extracted out of the liquid of whole apples and/or apple juice extraction residues treated with ethanol.

According to claim 3, it is possible to extract ceramide-containing matter from the liquid of whole apples and/or apple juice extraction residues treated with ethanol, which matter results in an improvement of memory impairment and/or a rise of the phosphorylation level of extracellular signal-regulated kinase (ERK1/2).

According to claim 4, ursolic acid can be extracted as Product 2 out of the liquid of whole apples and/or apple juice extraction residues treated with ethanol.

According to claim 5, it is possible to recover ethanol from the liquid of whole apples and/or apple juice extraction residues treated with ethanol for reuse purposes.

According to claim 6, a polyphenol solution can be extracted as Product 3 from the liquid of whole apples and/or apple juice extraction residues treated with ethanol.

According to claim 7, ceramide can be separated and purified from ceramide-containing matter extracted out of the liquid of whole apples and/or apple juice extraction residues treated with ethanol.

According to claim 8, ceramide-containing matter and/or ursolic acid and/or a polyphenol solution extracted out of the liquid of whole apples and/or apple juice extraction residues treated with ethanol can be used as a raw material for foodstuffs and/or cosmetics.

According to claim 9, it is possible to obtain a composition that, upon administration, facilitates production of hyaluronic acid and/or type II collagen.

According to claim 10, it is possible to obtain that, upon administration, results in an improvement of memory impairment and/or a rise of the phosphorylation level extracellular signal-regulated kinase (ERK1/2).

### BRIEF EXPLANATION OF THE DRAWINGS

FIG. 1 depicts a stepwise method/process of extracting ceramide-containing matter as Product I from whole apples and/or apple juice extraction residues.

FIG. 2 depicts the effects of ceramide-containing matter on the production of hyaluronic acid by cultured cells derived from the human skin.

FIG. 3 depicts the effects of ceramide-containing matter on the expression of genes that produce hyaluronic acid by cultured cells derived from the human skin.

FIG. 4 depicts the effects of ceramide-containing matter on the production of type II collagen by cultured cells derived from the human cartilage.

FIG. 5 depicts the effects of ceramide-containing matter on the expression of genes that produce type II collagen by cultured cells derived from the human cartilage.

FIG. 6 depicts the effects of ceramide-containing matter on a memory impairment caused by NMDA receptor blocker MK-801.

FIG. 7 depicts the effects of ceramide-containing matter on the phosphorylation of ERK1/2 in neural model cells derived from rats.

FIG. 8 depicts the effects of ceramide-containing matter on the MEK/ERK signaling system in neural model cells derived from rats.

FIG. 9 depicts ceramide step 1 of treating an ethanol treatment liquid with an adsorbent.

FIG. 10 depicts ceramide step 2 of extracting ursolic acid a precipitate-free liquid and ursolic acid as Product 2 from an adsorbent-treated liquid.

FIG. 11 depicts ceramide step 3 of obtaining a concentrate and recovering ethanol from the precipitate-free liquid.

FIG. 12 depicts ceramide step 4 of extracting ceramide-containing matter as Product 1 and a polyphenol solution as Product 3 from the concentrate.

FIG. 13 depicts a step of obtaining ceramide from Product 1 or ceramide-containing matter by separation and purification.

FIG. 14 depicts a whole method/process of extracting such various useful ingredients as mentioned above from whole apples and/or apple juice extraction residues.

### MODES FOR CARRYING OUT THE INVENTION

Common step 1 in which whole apples and/or apple juice extraction residues are stirred with the addition of water followed by drying, and common step 2 in which ethanol is added to the whole apples and/or apple juice extraction residues subjected to water treatment and drying, and they are then stirred together for solid-liquid separation thereby fractionating them into an ethanol treatment liquid and post-ethanol treatment dried matter are similar to those set forth in the prior invention (see Japanese Patent No. 5902256 corresponding to Patent Document 1; Japanese Patent Document 2015-57378A). The method/process mode for carrying out the present invention comprises steps as mentioned below. These steps are carried out in order; however, each independent step may be repeated and then go to the next step. More specifically, the method/process according to the present invention comprises the following steps.

### Ceramide Step 1

An adsorbent is added to and stirred with the ethanol treatment liquid obtained in the aforesaid common step 2, after which the adsorbent is removed by solid-liquid separation to obtain the adsorbent treatment liquid.

### Ceramide Step 2

Solid matter precipitating by letting the aforesaid adsorbent treatment liquid standing is removed by solid-liquid separation.

### Ceramide Step 3

The aforesaid precipitate treatment liquid is concentrated under reduced pressures to obtain a precipitate and recover ethanol.

### Ceramide Step 4

A precipitate obtained by adding water to the concentrate and subjecting the concentrate to heat insulation is subjected to solid-liquid separation and then dried to extract ceramide-containing matter as Product 1.

Further, the following effects or advantages are obtained from the present invention:

5. Effect on the amount of hyaluronic acid produced by cells in the case where the ceramide-containing matter extracted as Product 1 in the aforesaid ceramide step 4 and dissolved in a solvent is added to a culture medium of human skin fibroblasts cells, and/or Effect on the amount of expression of genes taking part in production of hyaluronic acid as found by extracting genes (RNAs) from cells for comprehensive analysis of gene expression and real-time PCR analysis using a primer.
6. Effect on the amount of type II collagen produced by cells in the case where the ceramide-containing matter extracted as Product 1 in the aforesaid ceramide step 4 and dissolved in a solvent is added to a culture medium of human cartilage sarcoma cells, and/or Effect on the amount of expression of genes taking part in human cartilage sarcoma cell production as found by extracting genes (RNAs) from cells for comprehensive analysis of gene expression and real-time PCR analysis using a primer.
7. Effect on recovery from memory impairment by administration of a solution wherein Product I from the aforesaid ceramide step 4 is suspended, as demonstrated by the dementia model mice to be described later.
8. Effect on the activation of phosphorylation of a factor taking part in a cellular signaling pathway in the case where the ceramide-containing matter extracted as Product 1 in the aforesaid ceramide step 4 is dissolved in a solvent for administration, and Effect of the aforesaid signaling pathway on the aforesaid recovery from memory impairment.
9. Method/process where in the aforesaid ceramide step 2, solid matter precipitating upon the adsorbent-treated liquid let standing is subjected to solid-liquid separation and dried thereby extracting ursolic acid as Product 2.
10. Method/Process where in the aforesaid ceramide step 3, the precipitate-free liquid can be concentrated under reduced pressures for recovery of ethanol.
11. Method/Process wherein in the aforesaid ceramide step 4, a polyphenol solution is extracted as Product 3 in a solution portion in which a precipitate obtained by adding water to the concentrate followed by heat insulation (heat-retaining) is subjected to solid-liquid separation.
12. Method/Process in which ceramide is separated and purified from ceramide-containing matter extracted as Product 1 in the aforesaid ceramide step.

### EXAMPLE 1

The method/process step of extracting the ceramide-containing matter as Product 1 from an ethanol treatment liquid of a material obtained by water treatment and drying of whole apples and/or apple juice extraction residues will now be explained.

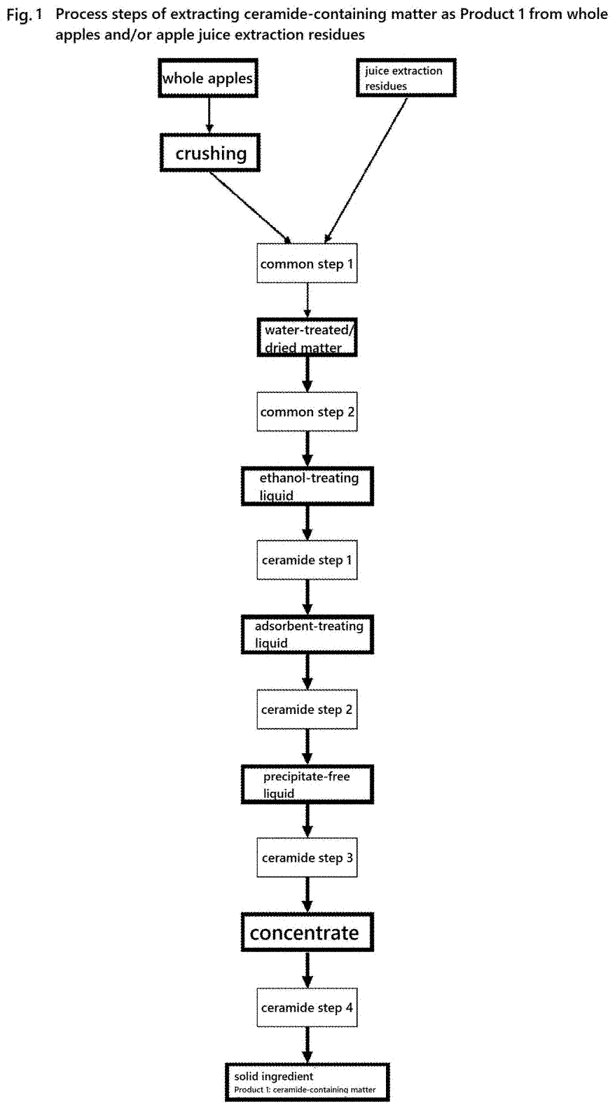

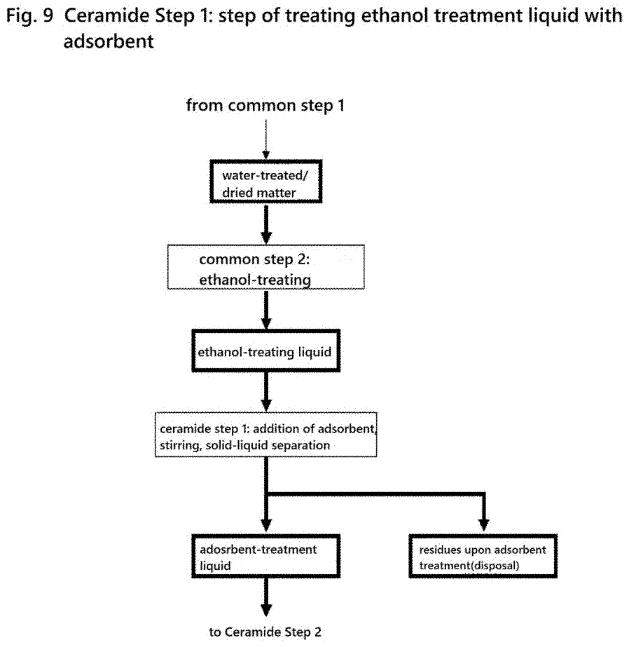

### Ceramide Step 1: Addition of an Adsorbent to the Ethanol Treatment Liquid (FIGS. 1, 9 and/or 14)

The adsorbent is added to and stirred with an alcohol treatment liquid of water-treated/dried material obtained in common step 1 (FIGS. 1 and/or 14), followed by separation into a liquid portion and the adsorbent by solid-liquid separation. This is called ceramide step 1. The adsorbent used herein may be active carbon, activate clay and so on. The amount of the adsorbent to be added may be 100 grams to 150 grams, preferably 120 grams per 1 kg of the initial residues upon water treatment/drying. The stirring time may be about 10 to 20 minutes, most preferably about 10 minutes at a stirring temperature between 40° C. and 70° C., preferably between 50° C. and 60° C. After stirring, a liquid portion is separated from the adsorbent as by centrifugation or suction filtration. This adsorbent is thrown away. The post-filtration liquid portion is let standing for one night while kept at 5° C. to 15° C.

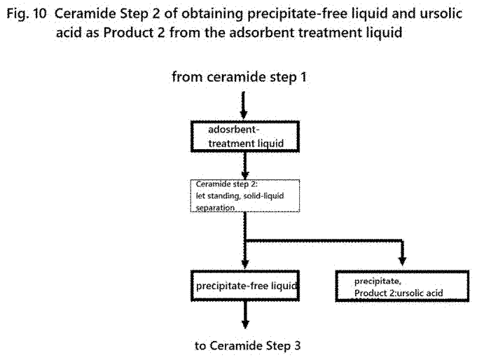

### Ceramide Step 2: Removal of Solid Matter Precipitating from the Adsorbent Treatment Liquid (FIGS. 1, 10 and/or 14)

Referring to a solution obtained by letting the adsorbent treatment liquid obtained from the aforesaid adsorbent treatment standing for one night, it is separated into a liquid portion and precipitates by a solid-liquid separation method such as centrifugation or suction filtration, preferably suction filtration using an analytical filter paper, because the precipitates settle down. The precipitate-free liquid obtained as a solution will be used in the next step.

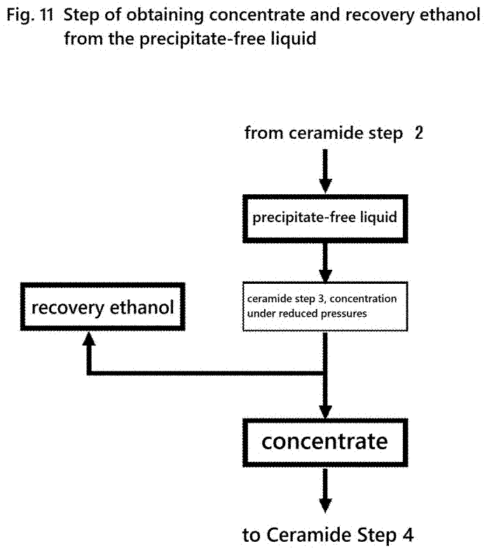

### Ceramide Step 3: Preparation of a Concentrate by Concentration of the Precipitate-free Liquid Under Reduced Pressures (FIGS. 1, 11 and/or 14

A concentrate is obtained by concentration under reduced pressures of the precipitate-free liquid obtained in ceramide step 2. This is called Ceramide Step 3 in which concentration is carried out with concentration means such as centrifugal thin-film vacuum evaporation equipment or a rotary evaporator, preferably under concentration conditions comprising heating at reduced pressures and temperatures no higher than the boiling point. The equipment needed for reduced pressures makes use of reduced pressure means such a diaphragm pump. If the necessary degree of reduced pressures (about 0.1 atm.) or the necessary degree of vacuum can be maintained, other evacuation means may also be used.

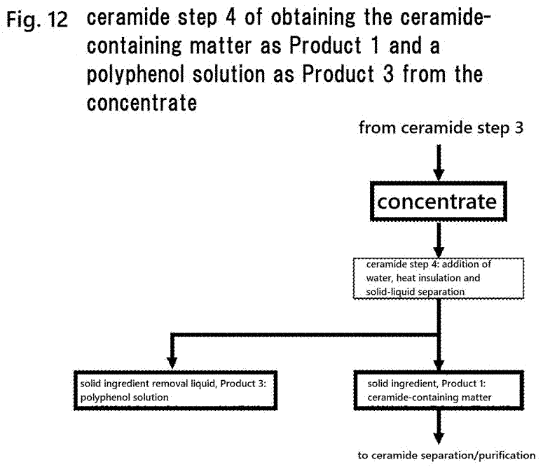

### Ceramide Step 4: Extraction of Ceramide-containing Matter as Product 1 by Adding Water to the Concentrate and Heat Insulation (Heat-retaining) (FIGS. 1, 12 and/or 14)

With water added to the concentrate obtained in ceramide step 3 and heat insulation, solid-liquid separation is carried out to separate a settling solid ingredient from an aqueous solution. This is herein called ceramide step 4. Water is added in an amount of 3 to 5 parts by volume, preferably 4 parts by volume per 1 part by volume of the concentrate. The stirring time is preferably about 10 to 50 minutes, more preferably 30 minutes, and the stirring temperature may be between 40° C. and 70° C., but preference is given to 50° C. to 60° C. The stirred aqueous solution, because of the solid component settling down therein, is subjected to solid-liquid separation, preferably suction filtration, for separation of the solid ingredient. The obtained solid ingredient is washed with warm water, and then dried under reduced pressure by desiccators. The solid ingredient referred to herein is Product I that is the ceramide-containing matter obtained in a yield of 1.5% to 2.0% as calculated on the basis of 1 kg of the original water-treated/dried residues.

Set out below are the results of extraction of the ceramide-containing matter as Product 1 by using the water-treated/dried matter as a starting material and carrying out ceramide steps 1 through 4.

(1) In ceramide step 1, the adsorbent was added to the ethanol-treated liquid followed by stirring, yielding an adsorbent-treated liquid.
(2) In ceramide step 2, the solid matter precipitating from the adsorbent-treated liquid was removed, yielding a precipitate-free liquid.
(3) In ceramide step 3, the precipitate-free liquid was concentrated under reduced pressure, yielding a concentrate.
(4) In ceramide step 4, water was added to the concentrate followed by heat insulation, yielding the ceramide-containing matter as Product 1 in a yield of 15 to 20 grams per 1 Kg of the residues.

### EXAMPLE 2

In Example 2, the effects of ceramide obtained from whole apples and/or apple juice extraction residues are shown with reference to the ceramide-containing matter extracted as Product 1 by way of ceramide steps 1 through 4 in Example 1.

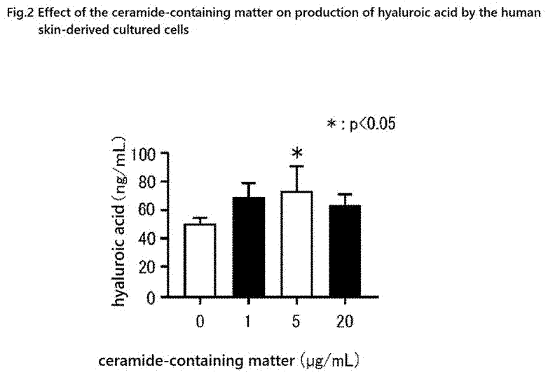

### Effect of the Ceramide-containing Matter on the Production of Hyaluronic Acid by Human Skin-derived Cultured Cells (FIG. 2)

Human fibroblasts (HSF) were cultured in a D-MEM culture medium containing 10% bobbin serum. The ceramide-containing matter extracted by way of the foregoing ceramide steps 1 through 4 was dissolved in dimethyl sulfoxide (DMSO), and the resulting solution was added to the cells in such an amount as to give 1 μg/mL, 5 μg/mL and/or 20 μg/mL for 72-hour culture, after which the supernatant liquid was recovered to quantify and estimate the amount of production of hyaluronic acid by the ELISA method, and the results are plotted in FIG. 2 with the concentration of the added ceramide-containing matter as abscissa and the amount of production of hyaluronic acid as ordinate. As the ceramide-containing matter was added in such an amount as to give 0.1 μg/mL and/or 5 μg/mL, it caused the amount of production of hyaluronic acid to increase so that when 5 μg/mL of the ceramide-containing matter were added, there was a significant difference found from the control.

### Comprehensive Analysis of the Ceramide-containing Matter Taking Part in Gene Expression by the Human Skin-deprived Cultured Cell

Human fibroblasts (HSF) were cultured in a D-MEM culture medium containing 10% bobbin serum. The ceramide-containing matter extracted by way of the foregoing ceramide steps 1 through 4 was dissolved in dimethyl sulfoxide (DMSO), and the resulting solution was added to the cells in such an amount as to give 5 μg/mL and/or 20 μg/mL. Twenty-four (24) hours after addition, all RNAs were extracted to use the next-generation sequencer for comprehensive analysis of transcription products by way of RNA sequence analysis of genes relating to skin care. As a result, type I collagen and type III collagen present abundantly in the skin had tended to increase, but not one of them increased significantly by a factor of 1.3 folds to 1.5 folds. On the other hand, when the ceramide-containing matter was added to HAS1 or a hyaluronic acid synthesis enzyme in an amount of 5 μg/mL and 20 μg/mL, there was a profound increase by a factor of 8.8 folds and 11.4 folds, respectively, and when the ceramide-containing matter was added HYAL1 or a hyaluronic acid-breaking enzyme in an amount of 5 μg/mL and 20 μg/mL, there was a decrease by a factor of 0.8 and 0.5 folds respectively.

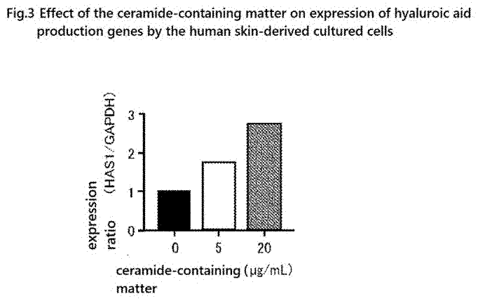

### Effect of the Ceramide-containing Matter on the Expression of Hyaluronic Acid Production Genes (FIG. 3)

Human fibroblasts (HSF) were cultured in a D-MEM culture medium containing 10% bobbin serum. The ceramide-containing matter extracted in the foregoing ceramide step 4 was dissolved in dimethyl sulfoxide (DMSO), and the resulting solution was added to the cells in such an amount as to give 5 μg/mL and/or 20 μg/mL to identify the expression of HAS1 or a hyaluronic acid synthesis enzyme gene by real-time PCR. The results are plotted in FIG. 3 with the concentration of the ceramide-containing matter added as abscissa and the amount of expression of hyaluronic acid production gene HAS1 as ordinate in term of a ratio to the amount of expression of GAPDH that were genes expressing in a constant amount commonly in housekeeping genes or cells. It has been understood that with reference to the cells with 5 μg/mL and 20 μg/mL of the ceramide-containing matter added thereto, respectively, the amount of expression of HAS1 is about two folds and about three folds more than the control, meaning that expression increases as is the case with the RNA sequence analysis.

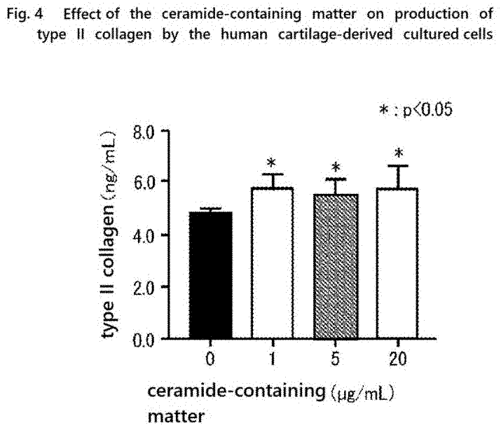

### Effect of the Ceramide-containing Matter on the Production of Type II Collagen by the Human Cartilage Derived Cultured Cell (FIG. 4)

The human cartilage sarcoma cell (OUMS27) was cultured in a D-MEM culture medium containing 10% bobbin serum. The ceramide-containing matter extracted in the foregoing ceramide step 4 was dissolved in dimethyl sulfoxide (DMSO), and the resulting solution was added to the cells in such an amount as to 1.0 μg/mL, 5 μg/mL and/or 20 μg/mL for 72-hour culture. After culture, the supernatant was recovered to quantify and estimate the amount of type II collagen produced by the ELISA method. The results are plotted in FIG. 4 with the amount of the ceramide-containing matter as abscissa and the amount of production of type II collagen. As the amount of the ceramide-containing matter increased from 1.0 μg/mL to 5 μg/mL and/or to 20 μg/mL, there was a tendency for the amount of production of type II collagen to increase.

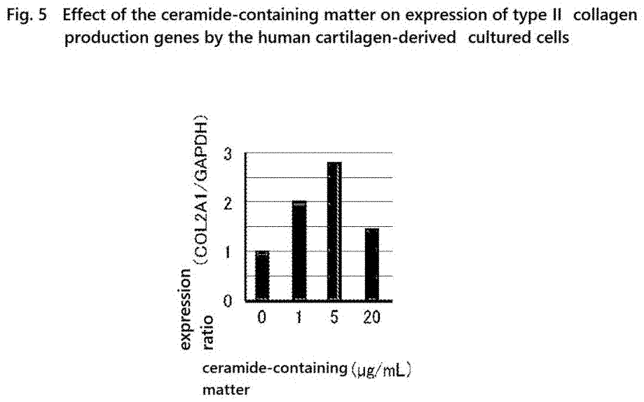

### Effect of the Ceramide-containing Matter on Gene Expression by the Human Cartilage-derived Cultured Cells (FIG. 5)

Upon the addition of the ceramide-containing matter extracted in the foregoing ceramide step 4 to a cultured solution of the human cartilage sarcoma cell (OUMS27), the results of gene expression of COL2A1 or type II collagen production genes are plotted in FIG. 5 with the concentration of the ceramide-containing matter as abscissa and the amount of expression of the type II collagen production genes as ordinate in terms of a ratio to the amount of expression of genes GAPDH expressing in a constant amount commonly in housekeeping genes or cells. In the cells added with the “ceramide-containing matter in an amount of 1.0 μg/mL and 5 μg/mL, respectively, the amount of expression of COL2A1 genes was almost doubled in the former and increased about 2.7 times in the latter.

The effects of the ceramide-containing matter set forth in Example 2 on the cultured cells derived from the human skin and/or cultured cells derived from the human cartilage are summarized below.

(1) The ceramide-containing matter induced the expression of hyaulronic acid production genes HAS1 in the cultured cells derived from the human skin fibroblasts to increase the production of hyaulronic acid.
(2) The ceramide-containing matter enhanced the expression of type II collagen production genes COL2A1 in the human cartilage cells, resulting in an increase in an amount of type II collagen production.

According to the present invention, therefore, it is possible to provide a composition capable of achieving such objects by using the ceramide obtainable from the whole apples and/or apple juice extraction residues as an effective ingredient for promotion or acceleration of hyaluronic acid and/or type II collagen. This may be applied not only to Product I of Example 1 but also to overall ceramides derived from apples.

### EXAMPLE 3

In Example 3, the effects of the ceramide obtained from whole apples and/or apple juice extraction residues on improvement of memory impairments and/or influences on an increase in the phosphorylation level of extracellular signal-regulated kinase (ERK1/2) are clarified typically with reference to the ceramide-containing matter extracted as Product 1 by way of ceramide steps 1 through 4.

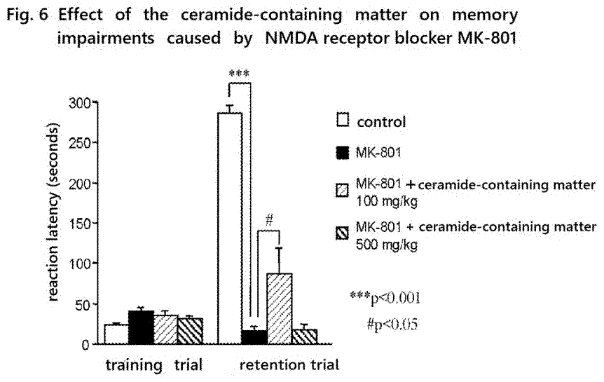

### Effect of the Ceramide-containing Matter on Memory Impairments Caused by N-methyl-D-aspartate Receptor Blocker MK-801 (FIG. 6)

N-methyl-D-aspartate (NMDA) receptor that is one of glutamic acid receptors is important for memory formation; administration of MK-801 that is a non-competitive NMDA receptor blocker to mice gives rise to memory impairments.

In this example, passive avoidance tests using dementia model mice having MK-801 administered thereto were carried out for estimation of effects of the ceramide-containing matter on memory impairments.

More specifically, 7 week old male DDY mice (available from Japan SLC, Inc.) were pre-fed for experimentation. The ceramide-containing matter suspended in an aqueous solution of 0.5% carboxymethyl cellulose sodium (CMC) was orally administrated to the mice once per day in a dose of 100 mg/kg body weight or 500 mg/kg body weight for six days. At the 7^{th} and 8^{th} days from the onset of administration, training and retention trials of passive avoidance testing were carried out. Ninety (90) minutes before the training trial of passive avoidance testing, 100 mg/kg body weight or 500 mg/kg body weight of the ceramide-containing matter or an aqueous solution of solvent 0.5% CMC were orally administrated to the mice, followed by subcutaneous administration of non-competitive NMDA receptor blocker MK-801 (0.2 mg/kg body weight) or a physiological saline solution 60-minute later. Thirty (30) minutes after that, training trial of passive avoidance testing was carried out, and after the elapse of 24 hours, retention testing was carried out.

The passive avoidance testing was carried out using Step-through type passive avoidance reaction equipment made up of a lighting room and a dark room through which electrical stimulation runs. First of all, mice were allowed to come in the lighting room as training trial, after which an electrical stimulation (0.4 mA, one second) was provided to them when the mice entered the dark room. Twenty-four (24) hours after that, the mice were again allowed to come in the lighting room as retention trial to measure the time until they entered the dark room (reaction latency) as a memory index.

The training trial used herein is understood to stand for a method/process in which the mice were allowed to come in the lighting room and then enter the dark room where they were allowed to receive an electrical stimulation. The retention trial used herein is understood to stand for a method/process of measuring a time taken to allow the mice to again enter the lighting room 24 hours after training trial and then the dark room as reaction latency. When the mice have a memory of receiving an electrical stimulation in the dark room, the reaction latency becomes long upon retention trial. Administration of the NMDA receptor blocker MK-801 to the mice induces a memory impairment to make the reaction latency so short that they move to the dark room. In the passive avoidance testing, the effect on an improvement of memory impairment is estimated with the reaction latency as an index.

The test results are shown in FIG. 6. In the retention trial, a group to which MK-801 alone was administered was significantly lower in the reaction latency than a control group to which the solvent was administered, indicative of memory impairment. On the other hand, a group to which 100 mg/kg body weight were administered was significantly longer in reaction latency than a group to which MK-801 alone was administered; the group to which the ceramide-containing matter was administered in a dose of 100 mg/kg body weight was clearly improved in terms of memory impairment induced by MK-801.

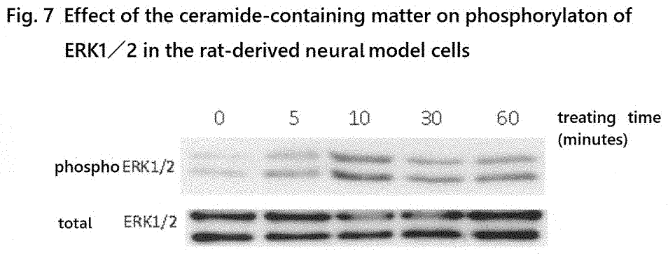

### Effect on the Phosphorylation of Extracellular Signal-regulated Kinase (ERK1/2) in Neural Model Cells (FIG. 7)

PC12 cells derived from rat adrenal gland pheochromocytoma are widely used as neural model cells. The PC 12 cells were cultured in a D-MEM culture medium containing 5% bovine serum and 5% horse serum. The ceramide-containing matter was dissolved in dimethyl-sulfoxide (DMSO), and the resulting solution was added to the cells in such a way as to give an amount of 30 μg/mL for culture for 5 minutes, 10 minutes, 30 minutes and 60 minutes. After culture, the cells were collected for estimation of the phosphorylation ERK1/2 (phospho ERK1/2) level important for memory formation by means of the western blot method using anti-phospho ERK1/2 antibody and/or anti-total ERK1/2 antibody. The results are shown in FIG. 7. By use of the ceramide-containing matter, there was observed a rise of the phosphorylation level of ERK1/2 with a peak found after the elapse of 10 minutes: the activation of ERK1/2.

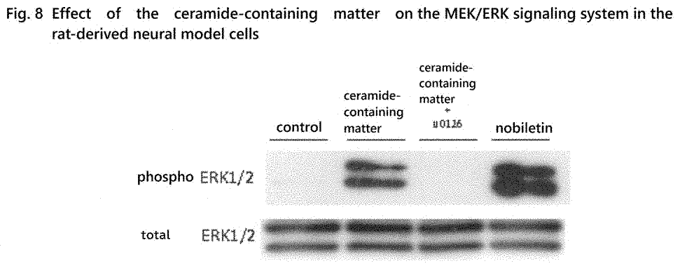

### Effect of the Ceramide-containing Matter on the MEK-ERK Signaling System in Neural Model Cells (FIG. 8)

An inhibitor of MEK that was the upstream kinase of extracellular signal-regulated kinase (ERK1/2) was used to make an estimation of the effect of the ceramide-containing matter on the MEK/ERK signaling system in PC-12 cells. The PC-12 cells were cultured in a D-MEM culture medium containing 5% bovine serum and 5% horse serum. The MEK inhibitor UO126 was dissolved in dimethyl-sulfoxide (DMSO), and the resulting solution was added to the cells in such an amount as to give 10 μM, and 30 minutes after that, the ceramide-containing matter dissolved in DMSO was added to the cells in such an amount as to give 30 μg/mL for 10-minute culture. After culture, the cells were collected for estimation of the phosphorylation ERK1/2 (phospho ERK1/2) level important for memory formation by means of the western blot method using anti-phospho ERK1/2 antibody and/or anti-total ERK1/2 antibody. The results are shown in FIG. 8. Note here that a sample treated with nobiletin (30 μM) was used as a positive control. By use of the ceramide-containing matter, the ERK1/2 phosphorylation level was observed to rise, but that rise was kept low by the pretreatment with the MEK inhibitor UO126, indicating that the ceramide-containing matter activates the MEK-ERK signaling system.

Set out below are the effects of the ceramide-containing matter on the dementia model mice and/or the neural model cells disclosed in Example 3.

(1) The ceramide-containing matter could result in improvements of memory impairments in dementia model mice to which N-methyl-D-aspartate (NMDA) receptor blocker MK-801 was administered.
(2) The ceramide-containing matter could result in a rise in the phosphorylation level of extracellular signal-regulated kinase (ERK1/2) important for memory formation in neural model cells or PC12 cells: the activation of ERK1/2.

According to the present invention, therefore, it is possible to provide a composition containing ceramide(s) obtained from whole apples and/or apple juice extraction residues as an effective ingredient for improving memory impairments and/or increasing the phosphorylation level of extracellular signal-regulated kinase (ERK1/2). It is here noted that the foregoing may be applied to not only Product 1 of Example 1 but also the general spectrum of ceramides derived from apples.

### EXAMPLE 4

Set out in Example 4 are ursolic acid as Product 2 and/or recovered ethanol and/or a polyphenol solution as Product 3, all obtained in intermediate steps comprising the aforesaid ceramide steps 2, 3 and 4.

### Ceramide Step 2 of Extracting Ursolic Acid as Product 2 from the Adsorbent Treatment Liquid (FIGS. 1, 10 and/or 14)

Product 2 was extracted by means of centrifugation or solid-liquid separation using suction filtration, preferably an analytical filter paper, because precipitates settled down in a solution stemming from the adsorbent treatment liquid obtained from the adsorbent treatment and let standing for one night. The yield of Product 2 is 0.9% to 1.5% as calculated on the basis of 1 kg of the original water treatment/dried residues.

### Analysis of Ursolic Acid by Thin-layer Chromatography

Product 2 extracted in the foregoing ceramide step 2 was dissolved in a solvent comprising a 4:1 chloroform/methanol mixture solvent, and the resulting solution was coated on a silica gel thin layer to carry out thin-layer chromatography using a 65:25:4 chloroform/methanol/water mixture solvent as a developing solvent, after which a copper/phosphoric acid coloration reagent was sprayed on the surface of the silica gel thin layer and then heated for coloration. As a result, it has been found that Product 2 precipitated and extracted in ceramide step 2 is ursolic acid.

### Ceramide Step 3: Recovery of Ethanol by the Concentration under Reduced Pressures of the Precipitate-free Liquid Obtained in Ceramide Step 2 (FIGS. 1, 11 and/or 14)

The precipitate-free liquid obtained in ceramide step 2 can be concentrated under reduced pressures to obtain a concentrate and recover ethanol that may be reused. The recovered ethanol may be reused as ethanol in ceramide step 1. In a typical example, the ethanol recovery rate was 70%. As a result of measuring the water content of recovered ethanol by means of a density indicator, it has been found to keep the original water content intact, meaning that the ethanol may be reused with no need for any additional step.

### Ceramide Step 4: Preparation of a Polyphenol Solution of Product 3 by Water Addition and Heat Insulation of the Concentrate (FIGS. 1, 12 and/or 14)

In ceramide step 4, water is added to the concentrate obtained in ceramide step 3 followed by thermal insulation for solid-liquid separation of a precipitating solid ingredient from an aqueous solution. The liquid portion is polyphenol-containing matter. In a typical example, 0.5 liter of the concentrate was added with water in a 4-fold amount or 2 liters, and the obtained precipitate was washed with 2 liters of fresh water, whereupon the filtrate was obtained as a polyphenol solution in a combined amount of 4.5 liters.

### Analysis of the Polyphenol Solution Obtained in Ceramide Step 4

In ceramide step 4, a polyphenol solution is obtained as a solution portion in which the ceramide-containing matter precipitating after water addition and heat insulation is subjected to solid-liquid separation. As a result of measuring the content of polyphenol contained in this solution by the Folin-Denis method, it has been found that the amount of polyphenol contained in 100 mL solution is 150 mg as calculated as the amount of catechin. Therefore, the polyphenol solution had a concentration of 0.15%, and was obtained in an amount of 6.75 grams from 1 Kg of the original water/drying treatment residues.

As set forth in Example 4, the results obtained in the respective steps from ceramide step 2 to ceramide step 4 are summarized below.

(1) In ceramide step 2, ursolic acid was extracted as Product 2 from the adsorbent-free liquid in an amount of 0.9% to 1.5% per 1 Kg of the original water-treated/dried matter.
(2) In ceramide step 3, the concentrate could be prepared with ethanol recovery in a recovery rate of 70%.
(3) In ceramide step 4, the polyphenol solution was obtained in a solution from which a solid ingredient precipitating by water addition and heat insulation of the concentrate in a volume of 4.5 L per 1 Kg of the residues with a polyphenol concentration of 150 mg/100 mL as calculated on a catechin basis.

### EXAMPLE 5

In Example 5, the ceramide-containing matter extracted in the foregoing ceramide step 4 was subjected to separation/purification and instrumental analysis.

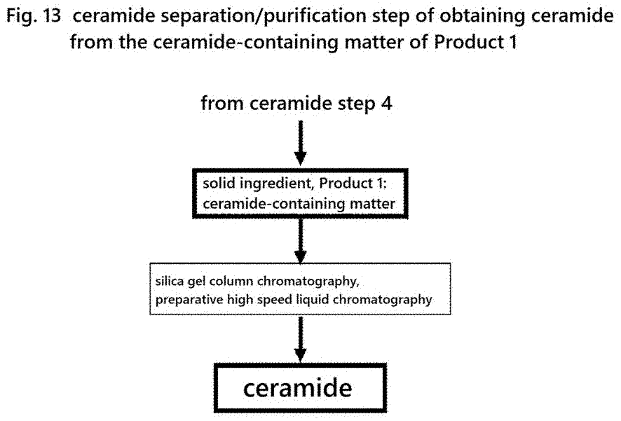

### Separation/Purification of the Ceramide-containing Matter by Silica Gel Column Chromatography (FIGS. 13 and/or 14)

Zero point two (0.2) gram of the ceramide-containing matter extracted in the foregoing ceramide step 4 was taken and dissolved in 2 mL of a solvent in which chloroform and methanol were mixed in 4:1. Then, silica gel suspended in a 4:1 chloroform/methanol mixture solvent, preferably 230 to 400 mesh silica gel available from Merck & Co. was filled up in a glass column tube having a diameter of 30 mm in such a way as to provide a height of 420 mm. Note here that the column volume was about 300 mL. A solution in which the foregoing ceramide matter was dissolved was adsorbed onto the tip of the silica gel column, and a 4:1 chloroform/methanol mixture solvent was allowed to flow down the column in a flow rate of 2 mL/min. Then, a solution flowing out of the lower end of the column was subdivided in test tube Nos. 1 to 40, each in an amount of 10 mL. Of these test tubes, test tube Nos. 15 to 34 were each spotted on a silica gel thin-layer plate (silica gel 60F254 of 10 cm×20 cm available from Merck & Co., and then developed using a developing solvent comprising a 65:25:4 chloroform/methanol/water mixture solvent. After development, an anthrone sulfate reagent was sprayed and heated for coloration. The thin-layer plate was spotted at both its ends with a commercially available ceramide standard (derived from soy beans as a standard substance for comparison of mobility and color tone. Ceramide was separated from test tube No. 31 to No. 33 by column chromatography of the ceramide-containing matter.

### Separation and Purification of Ceramide by Preparative High Speed Liquid Chromatography (FIGS. 13 and/or 14)

The content of Test tube No. 33 collected from test tube No. 31 subjected to the foregoing separation by silica gel column chromatography was concentrated under reduced pressure, and dissolved in a 95:5 methanol/water mixture solvent. The preparative high speed chromatography was carried out by connecting a high speed liquid chromatography system with a preparative reversed chromatography system, preferably TSKGELODS-80TS (available from TOSOH CORPORATION) having a column size of 8 mm in diameter and 300 mm in length, and using as an eluent a 95:5 methanol/water mixture solvent at a flow rate of 2 mL/min. for detection at 210 nm. An ingredient flowing out at a holding time of 19.96 minutes was collected and concentrated to dryness for instrumental analysis.

### Analysis of Ceramide by Infrared Adsorption Spectra

Measured here were the infrared adsorption spectra of the ingredient flowing out at the holding time of 19.96 minutes by the foregoing preparative high speed liquid chromatography, and for the infrared adsorption spectra, a sample was regulated according to the KBr-Disc method, and a Fourier transform infrared analyzer was then used to measure transmittances (T%) from 4000 cm-1 to 400 cm-1. The spectra conformed to those of the ceramide.

### Analysis of Ceramide by Nuclear Magnetic Resonance Spectra

An ingredient flowing out at the holding time of 19.96 minutes in the foregoing preparative high speed liquid chromatography was dissolved in a solution comprising a 4:1 heavy chloroform/heavy methanol mixture, and nuclear magnetic resonance spectra were measured using a 270 MHz Fourier transform nuclear resonance device (available from JEOL) with tetramethylsilane as the internal standard.

Set out below are the results of separation/purification or instrumental analysis of ceramide contained in the ceramide-containing matter disclosed in Example 5.

(1) The ceramide was separated from the ceramide-containing matter and purified by silica gel column chromatography followed by preparative high speed liquid chromatography.
(2) The structure of the ceramide separated and purified by the foregoing chromatography means are analyzed by infrared adsorption spectra and/or nuclear magnetic resonance spectra. These spectra conformed to those of the ceramide.

### APPLICABILITY TO THE INDUSTRY

The present invention provides an extraction method/process in which matter or material obtained by water treatment and drying of whole apples and/or apple juice extraction residues is used as the raw or starting material, and then treated with ethanol thereby extracting ceramide-containing matter or ceramide and/or ursolic acid and/or a poly-phenol solution contained in the resulting ethanol treatment liquid. In this process, ingredients contained in the ethanol treatment liquid can be utilized, and the ethanol used for extraction can be recovered and reused. By the present invention it is possible to use whole apples thrown away without being eaten raw or processed as a raw material, resulting in industrial use of apple juice resides that are discarded as unavailable resource or apply loads to the environment.

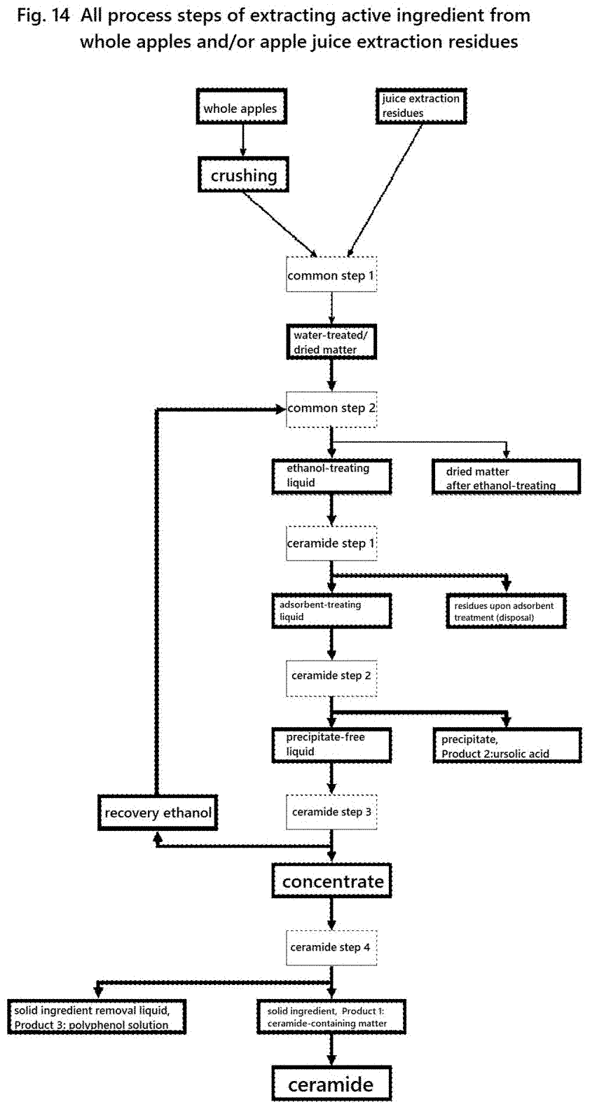

In the present invention, it has been verified on substance and gene expression levels that the ceramide-containing matter extracted at the foregoing respective method/process steps increases or enhances production of hyaluronic acid by the human skin culture cells and production of type II collagen by the human cartilage cells. Human-derived hyaluronic acid or type II collagen is a substance that has an important role in each human tissue and a direct effect on maintenance of health and appearance or improvements in the quality of life. It has been found for the first time that the ingredients contained in whole apples and/or apple juice resides have an effect on enhancement of production of hyauronic acid or type II collagen. Whole apples and/or apple juice extraction residues are available in abundance, and the useful ingredients obtained in the process steps of FIGS. 1 through 14 can be industrially utilized as foodstuff material or cosmetics material.

In the present invention, the ceramide-containing matters extracted in the respective process steps brought about an improvement in memory impairments in dementia model mice and a rise of the level of phosphorylation of extracellular signal-regulated kinase (ERK1/2) important for memory formation in rat-derived neural model cells: activation of ERK1/2. Improvements of dementias or memory impairments have a direct influence on maintenance of health or an improvement in the quality of life with much more patients in the aging society in mind. It has been found for the first time that the ingredients contained in whole apples and/or apple juice extraction residues have an effect on improvements of dementias and memory impairments. Whole apples and/or apple juice extraction residues are available in abundance; useful ingredients obtained in the respective process steps of FIGS. 1 to 14 can be used in an industrial scale as materials for health foodstuff and pharmaceuticals.

1. A method for extracting ceramide-containing matter comprising:
a ceramide step 1 wherein whole apples or apple juice extraction residues are treated with water and then dried to obtain a sample, then the sample is added with ethanol and stirred for separation into an ethanol treatment liquid and post-ethanol treatment dried matter, and the resulting ethanol treatment liquid is added and stirred with an adsorbent, after which the adsorbent is removed by solid-liquid separation to obtain an adsorbent treatment liquid,
a ceramide step 2 wherein deposits precipitating by letting the adsorbent treatment liquid standing are separated to obtain a precipitate-free liquid,
a ceramide step 3 wherein the precipitate-free liquid obtained from the ceramide step 2 is concentrated under reduced pressures to obtain a concentrate, and
a ceramide step 4 wherein a solid ingredient settling down by adding water to the concentrate obtained from the ceramide step 3 followed by heat insulation is separated and dried to obtain ceramide-containing matter as Product 1.

2. A method for extracting ceramide-containing matter as recited in claim 1, wherein, upon administration, the ceramide-containing matter obtained as Product 1 by way of the respective ceramide steps 1 through 4 brings about a significant rise of production of hyaluronic acid or a significant rise of production of type II collagen.

3. A method for extracting as recited in claim 1, wherein, upon administration, the ceramide-containing matter obtained as Product 1 by way of the respective ceramide steps 1 through 4 contributes to an improvement of memory impairment or a rise of phosphorylation level of extracellular signal-regulated kinase (ERK1/2).

4. A method for extracting ceramide-containing matter as recited in claim 1, wherein ursolic acid is precipitated as Product 2 in the ceramide step 2.

5. A method for extracting ceramide-containing matter as recited in claim 1, wherein ethanol is collected in the ceramide step 3 for reuse as the ethanol in the ceramide step 1.

6. A method for extracting ceramide-containing matter as recited in claim 1, wherein a poly-phenol solution is obtained as Product 3 in the ceramide step 4.

7. A method for extracting ceramide comprising:
a ceramide step 1 wherein whole apples or apple juice extraction residues are treated with water and then dried to obtain a sample, then the sample is added with ethanol and stirred for separation into an ethanol treatment liquid and post-ethanol treatment dried matter, and the resulting ethanol treatment liquid is added and stirred with an adsorbent, after which the adsorbent is removed by solid-liquid separation to obtain an adsorbent treatment liquid,
a ceramide step 2 wherein deposits precipitating by letting the adsorbent treatment liquid standing are separated to obtain a deposit-free liquid,
a ceramide step 3 wherein the precipitate-free liquid obtained from the ceramide step 2 is concentrated under reduced pressures to obtain a concentrate,
a ceramide step 4 wherein a solid ingredient settling down by adding water to the concentrate obtained from the ceramide step 3 followed by heat insulation is separated and dried to obtain ceramide-containing matter as Product 1, and
a step of obtaining ceramide from the ceramide-containing matter.

8. Foodstuffs, or a cosmetic material containing the ceramide-containing matter obtained by the method for extracting ceramide set forth in claim 1.

9. A composition for facilitation of hyaluronic acid and/or type II collagen, which contains ceramide obtained from whole apples or apple juice extraction residues.

10. A composition for facilitation of an improvement of memory impairment or a rise of phosphorylation level of extracellular signal-regulated kinase (ERK1/2), which contains ceramide obtained from whole apples or apple juice extraction residues.

11. Foodstuffs, or a cosmetic material containing ursolic acid obtained by the method for extracting ceramide set forth in claim 4.

12. Foodstuffs, or a cosmetic material containing the polyphenol solution obtained by the method for extracting ceramide set forth in claim 6.
